(19) United States
(12) Patent Application Publication
(10) Pub. No.: US 2021/0128023 A1
(43) Pub. Date: May 6, 2021

(54) Configurable Fashion Accessory Including an Electronic System for Activity Tracking

(71) Applicant: Liliana Kennedy-Paesler, Austin, TX (US)

(72) Inventor: Liliana Kennedy-Paesler, Austin, TX (US)

(21) Appl. No.: 17/085,034

(22) Filed: Oct. 30, 2020

Related U.S. Application Data
(60) Provisional application No. 62/928,914, filed on Oct. 31, 2019.

(51) Int. Cl. A61B 5/11; A61B 5/00; A61B 5/024; A61B 5/01

(52) U.S. Cl. CPC A61B 5/1118; A61B 5/6803; A61B 5/0004; A61B 2560/0214; A61B 5/024; A61B 5/01; A61B 5/0015

(57) ABSTRACT

An activity tracking fashion-accessory kit includes an electronic system configured to measure parameters concerning physical activity when worn by a user and a plurality of resilient bands each including an enclosure configured to temporarily receive the electronic system in a manner that secures the electronic system within the resilient band, respectively. Each one of the respective plurality of resilient bands are sized and configured for use in each of a first configuration as a wrist band and a second configuration as a hair tie. The electronic system is housed in a housing that interchangeably fits within each of the respective enclosures, and configured to track a step count of the user when enclosed in any of the respective enclosures with the respective resilient band worn by the user in each of the first configuration and the second configuration, respectively.

### BACKGROUND OF INVENTION

### 1. Field of Invention

This invention relates generally to apparatus, systems and methods for activity tracking. More specifically, at least one embodiment relates to a configurable fashion accessory including an electronic system for activity tracking.

### 2. Discussion of Related Art

Wearable activity trackers have become a ubiquitous consumer electronic device. Individuals use activity trackers to monitor their physical training, to monitor their general level of activity and to monitor the status of physiological conditions known to be important identifiers of a healthy lifestyle and/or a heightened risk of a health issue. Dedicated activity tracking devices are typically worn by the user in some fashion, for example, worn around the wrist. Today's smart watches also include functions typically associated with activity trackers. As a result, users must generally dedicate “real estate” on their wrist to wear conventional activity trackers. The preceding is less than desirable because activity trackers and smart watches lack the stylized features of a conventional watch or other fashion accessory. Instead, today's activity trackers have the less stylized appearance of a consumer electronic device. The appearance of many activity trackers actually makes them unsuitable for certain venues and formal events, for example, weddings. This restricts the ability of users to continuously monitor their activity and/or health. Because current activity trackers are conspicuous they make it clear that the user is tracking their activity. This is a drawback when the user wishes to be more discreet regarding their activity tracking.

Current activity trackers also lack an ability to provide meaningful metrics for many common exercises used in physical training. For example, a wrist-worn activity tracker may not accurately measure motion for activities such as pushups, crunches, squats, and other movements common in yoga, bane, Pilates, and dance. The preceding results because the part of the user's anatomy where the activity tracker is worn during the activity either doesn't move or has a very limited range of motion that is not indicative of the level of user-activity even where the movement is measureable.

In addition to the preceding drawbacks, today's activity trackers are typically only suited for wear on an appendage such as the wrist or at some other location that is offset from the spine of the user. The offset (or misalignment) from the anatomical “central line” of the user creates challenges in accurately sensing and measuring a user's activity. Further, today's activity trackers lack a means to accurately monitor a user's posture and/or the tilt of their head. Finally, hair ties are a high utility accessory for people with long hair. Despite there being many options for hair ties on the market, most designs struggle to hold hair securely without damaging it while also becoming worn out and fragile over time.

### SUMMARY OF INVENTION

Therefore, there is a need for apparatus, systems and methods that allow an activity tracking device to be worn without notice while also increasing the accuracy of measurements made by the activity tracking device. According to some embodiments, an activity tracker is integrated in an otherwise conventional hair tie. According to these embodiments, the hair-tie activity tracker measures a user's activity with the device located in a location that is substantially aligned with the central line of the user. This location can improve the accuracy and consistency of the values that are measured and/or derived from the sensor data record by the device. These embodiments can be worn in public without notice while freeing up the user's wrist or any other location typically employed to wear an activity tracker. In addition to the preceding, the hair-tie activity tracker allows for sensing the user's posture and/or the tilt of their head. Embodiments can also accurately measure activities such as pushups, crunches, squats, inversion, and other movements common in yoga, bane, Pilates, and dance. As a result, these embodiments can provide meaningful metrics for these types of movements. The preceding can be achieved because meaningful measurements concerning the user's activity can be provided with an activity tracking system that measures a movement of the user's head. Also, movement of the torso and in some cases associated lower body movement (for example, with the user doing pushups) can be accurately tracked with the activity tracker worn as a hair tie. In addition to tracking movement and posture, embodiments can also provide a hair tie with structure that holds hair more effectively and securely than conventional hair ties. The hair tie is also more durable than existing options as a result of a selection of the materials of construction. In further embodiments, the activity tracking fashion accessory is provided with an overall tubular lattice structure including a pocket or other enclosure that receives the electronic system. In general, the embodiments described herein can be employed for activity tracking including fitness tracking.

According to one aspect, a system configurable for wear by a user in a plurality of locations for activity-tracking is provided. The system includes a resilient member having an overall shape that is substantially ring-shaped when in a relaxed state and configurable in a plurality of states including the relaxed state and a plurality of different stretched states. According to these embodiments, the resilient member is sized with an inner diameter smaller than a width of user's hand when in the relaxed state. The electronic system is housed for secure attachment to the resilient member with the resilient member in any of the plurality of states. The electronic system is configurable in a first operating mode for measuring with the resilient member worn on a wrist of the user at least one physiological parameter and at least one parameter concerning a movement of the user. The electronic system is configurable in a second operating mode for measuring with the resilient member worn as a hair tie by the user at least two of the following parameters: the at least one physiological parameter; the at least one parameter concerning the movement of the user; and at least one parameter concerning a posture of the user.

According to some embodiments, the resilient member is deformable to place the resilient member into each of the plurality of different stretched states such that the overall shape of the resilient member in each the plurality of different stretched states includes non-circular shapes having inner diameters with dimensions that differ from the inner diameter of the resilient member in the relaxed-state, respectively. The electronic system is configured to receive at least one user input concerning a location on the user where the resilient member is secured. According to some further embodiments, the electronic system is configured to automatically record data measured for the at least one parameter concerning the posture of the user when the at least one user input indicates that the resilient member is being worn as the hair tie and automatically suspend a recording of data measured for the at least one parameter concerning the posture of the user when the at least one user input indicates that the resilient member is being worn as a wrist band.

According to another aspect, an activity tracking fashion-accessory kit is provided. The fashion-accessory kit includes an electronic system is configured to measure parameters concerning physical activity when worn by a user and a plurality of resilient bands each including an enclosure configured to temporarily receive the electronic system in a manner that secures the electronic system within the resilient band, respectively. According to some embodiments, each one of the respective plurality of resilient bands is sized and configured for use in each of a first configuration as a wrist band and a second configuration as a hair tie. The electronic system is housed in a housing that interchangeably fits within each of the respective enclosures, and configured to track a step count of the user when enclosed in any of the respective enclosures with the respective resilient band worn by the user in each of the first configuration and the second configuration, respectively.

According to a further aspect, a method of tracking a user's activity with a fashion accessory is provided. According to some embodiments, the method includes locating an activity tracking electronic system within a housing, the electronic system configured to wirelessly transmit sensor data concerning a movement of a user wearing the activity tracking electronic system to a portable electronic device in the user's possession and providing a fashion accessory for use in each of a first configuration as a wrist band and a second configuration as a hair tie, the fashion accessory including an enclosure having an opening sized and configured to receive the housing. According to a further embodiment, the method includes acts of: inserting the housing within the enclosure via the opening; in the first configuration, securing the fashion accessory around a wrist of the user in a manner that blocks both the opening and the housing from view; and in the second configuration, securing the fashion accessory around a bundle of hair in a manner that blocks both the opening and the housing from view. According to other embodiments, the housing may be directly accessible to the user to allow them to make contact with the housing, for example, for the purpose of a touch sensitive measurement such as a measurement concerning pulse, heart rate, EKG or body temperature. According to other embodiments, the accessibility allows the user to measure physiological parameters in a touchless manner by placing their finger adjacent the housing without touching the housing, for example, while they are wearing the fashion accessory. Examples include allowing a user wearing the fashion accessory as a hair tie to reach behind their head and measure their heart rate using a photoplethysmography (PPG) sensor included in the electronic system. According to some of these embodiments, the housing is visible when the fashion accessory is being worn by the user.

As used herein, the term “posture” refers to a position of any one of or any combination of the head, the spine or the torso relative to a reference. For example, those of ordinary skill in the art in view of the disclosure provided herein will recognize that the term posture refers to a tilt of the head in any direction relative to vertical, a tilt of the head in any direction relative to the spine, a position of the spine relative to vertical and a position of the spine relative to the pelvis. According to the preceding, as used herein, the phrase “parameter concerning a posture of the user” concerns a value indicative of the any one or any combination of the preceding. In general, posture can include changes in an angle including relative to an anatomical plumb line and/or a rotational position such as a rotation of the head relative to the spine or the torso relative to the pelvis as some examples. One of ordinary skill in the art will also recognize in view of the disclosure provided herein that a movement of the arms is not a change in posture as the term is used herein.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates a device for activity tracking in accordance with a first embodiment;

FIG. 2 illustrates a device for activity tracking in accordance with a second embodiment;

FIG. 3 illustrates a device for activity tracking in accordance with a third embodiment;

FIG. 4 illustrates a device for activity tracking in accordance with a fourth embodiment;

FIGS. 5A & 5B illustrate a device for activity tracking in accordance with a fifth embodiment;

FIG. 6 illustrates a block diagram of an electronic system for inclusion in a device for activity tracking in accordance with various embodiments;

FIG. 7 illustrates a system including a device for activity tracking in accordance with one embodiment; and

FIG. 8 illustrates a device for activity tracking in accordance with a sixth embodiment;

### DETAILED DESCRIPTION

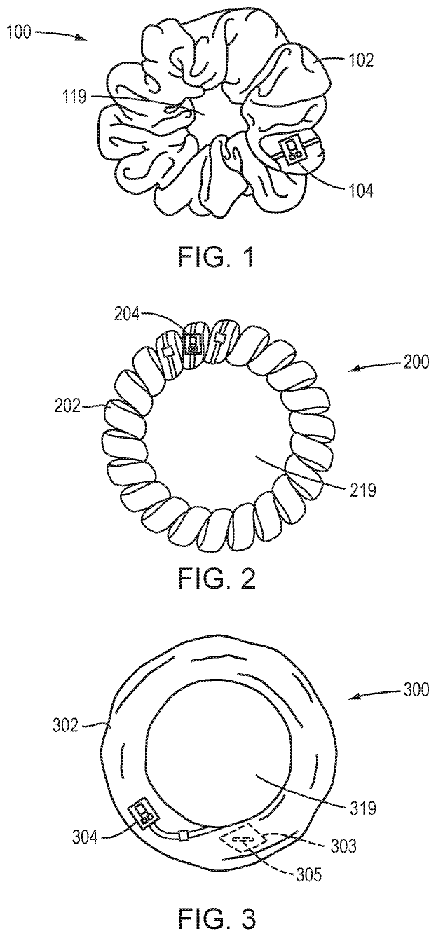

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. Referring to FIG. 1, a device 100 for activity tracking is illustrated in accordance with a first embodiment. The device 100 includes a resilient band 102 and an electronic system 104. According to one embodiment, the device 100 is a scrunchie-style hair tie that includes an outer fabric layer and an elastic band enclosed by the outer fabric. The electronic system 104 can be located beneath the outer fabric layer or visible on the exterior of the fabric depending on the embodiment. For example, where the electronic system 104 is located beneath the fabric, the outer layer of fabric can include a closure using VELCRO, a button, snap or other fastener to temporarily seal an opening through which the electronic system 104 is accessed, when necessary.

In some embodiments, the device 100 is configured as an activity tracking fashion accessory that can be used in multiple configurations. For example, the device 100 can be worn as a hair tie in a first configuration and as a wrist band in a second configuration. In the first configuration, the device 100 is wrapped around a bundle of the user's hair, for example, to create a pony tail. As is described in greater detail below, the device 100 operates as an activity tracking device when worn in this first configuration. The device 100 includes a central opening 119 that is sized to allow the device to be worn interchangeably in both configurations. For example, according to some embodiments, the diameter of the central opening 119 is sized to be smaller than a diameter of the user's hand. According to this embodiment, the resilient band 102 is stretched to slide the device over the user's hand and onto their wrist. The diameter of the central opening is sized to allow the device 100 to be worn in this fashion without sliding off during the user's activity. The device 100 is configured to operate as an activity tracker when worn around the wrist in this second configuration. The user can reverse the process and stretch the band over the hand for removal and then reuse the device 100 as a hair tie operating as an activity tracker when secured around a bundle of their hair.

Referring to FIG. 2, a device 200 for activity tracking is illustrated in accordance with a second embodiment. The device 200 includes a resilient band 202 and an electronic system 204. The device 200 is provided in the form of a circular coiled cord, sometimes referred to as a “spiral” design. In the illustrated embodiment, the resilient band 202 has an appearance similar to that of a coiled phone cord. In some embodiments, the resilient band is manufactured from plastic or rubber or a plastic-rubber hybrid. The type of material with which the resilient band 202 is manufactured can vary depending on the embodiment. For example, in one embodiment, the resilient band 202 is manufactured from polyurethane. The device 200 includes a central opening 219. According to some embodiments, the diameter of the central opening 219 is sized such that the device can be worn interchangeably as hair tie and as a wrist band while operating as an activity tracker.

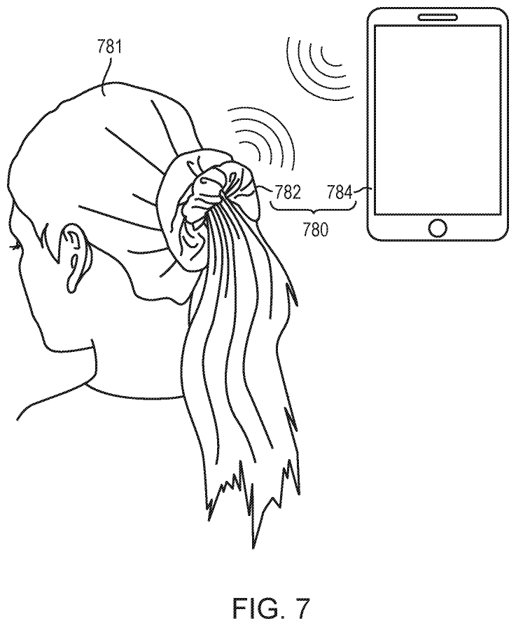

The configuration in which the device 200 is worn can impact the data measured by sensors included in the electronic system 204. For example, there is a difference in the dynamic movement of the device 200 during activity depending on whether the device is worn as a hair tie or a wrist band. In addition, there is a difference in a location of the electronic system 204 relative to a longitudinal central axis of the user or an anatomical plumb line, for example, depending on whether the device is worn as a hair tie or a wrist band. As is described in greater detail below, the device 200 is configurable to provide a high degree of accuracy when operating in either configuration. That is, the overall system in which the device 200 is employed is configurable to adjust the signal processing of the sensor outputs to maintain accuracy when the device 200 is worn in either configuration. Depending on the embodiment, the configurability can occur at the device 200, at a user's portable electronic device where signal processing is performed (see FIG. 7), at signal-processing resources accessed via the cloud or at any combination of the preceding. This approach is suitable for all embodiments described herein.

In one embodiment, the electronic system 204 is attached to opposite ends of the resilient band 202 to complete a 360 degree loop. That is, opposite ends of the resilient band 202 are attached to opposite ends of a housing including the electronic system 204 to complete the loop. According to another embodiment, the electronic system 204 is provided in a cylindrically-shaped housing having a diameter suitable to locate within the center of the coils of the resilient band 202. According to this embodiment, the housing includes flexible fasteners that can be wrapped around the cord to secure the housing including the electronic system 204 to the cord. The preceding secures the housing even when the coils are stretched out as the diameter of the cord is increased in the process of securing hair within it. According to still another embodiment, the electronic apparatus is included in a housing enclosed within the plastic material of the resilient band. According to this embodiment, an electrical connection included in the electronic system 204 penetrates the exterior surface of the resilient band 202. The connection can be used to connect a source of electrical power to recharge a power source such as a battery included in the electronic system 204 without removing the electronic system 204 from within the plastic.

Referring to FIG. 3, a device 300 for activity tracking is illustrated in accordance with a third embodiment. The device 300 includes a resilient band 302 and an electronic system 304. The device 300 also includes a central opening 319. According to some embodiments, the diameter of the central opening 319 is sized such that the device 300 can be worn interchangeably as hair tie and as a wrist band while operating as an activity tracker. In one embodiment, the electronic system 304 is located within the resilient band 302. According to another embodiment, the electronic system 304 is secured to the outer surface of the resilient band 302. In still another embodiment, the electronic system 304 is secured within the resilient band 302 with a portion of the system 304 exposed for user access, for example, to replace a battery or recharge the electronic system 304. In yet another embodiment, the electronic system 304 is attached to opposite ends of the resilient band 302 to complete a 360 degree loop. That is, opposite ends of the resilient band 302 are attached to opposite ends of a housing including the electronic system 304 to complete the loop. In some embodiments, the resilient band 302 includes a textured outer surface, for example, ridges, bumps or other surface variations, to assist in securing hair within the device 300.

According to another embodiment, the resilient band includes an enclosure 303 located within the resilient band and an opening 305, for example, a slit through which a housing including the electronic system 304 is inserted to locate the electronic system 304 within the enclosure 303 in an interior of the resilient band 302. According to this embodiment, the electronic system 304 can be located to place it out of site. This option allows the user to inconspicuously wear the activity tracking electronic system 304 on either their wrist or as a hair tie as a fashion accessory. In FIG. 3, the enclosure 303 and opening 305 are illustrated in phantom because they may be optionally employed in a given embodiment.

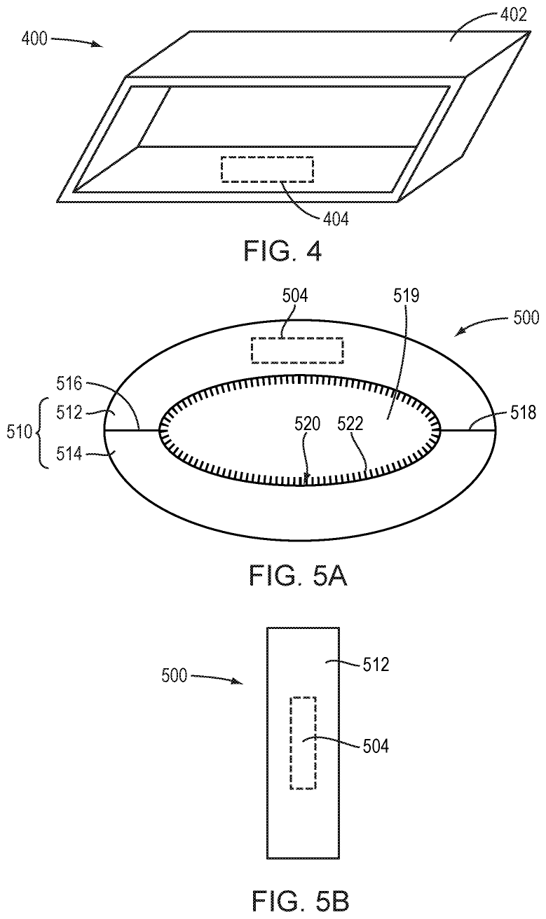

Referring to FIG. 4, a device 400 for activity tracking is illustrated in accordance with a fourth embodiment. The device 400 includes a deformable band 402 and an electronic system 404. According to this embodiment, the device 400 is a flexible, non-resilient hair tie. That is, the device 400 bends but doesn't stretch in use. Accordingly, the deformable band 402 is pressed into the desired shape once the user's hair is fed through the device 400. In one embodiment, the electronic system 404 is located within the deformable band 402. According to another embodiment, the electronic system 404 is secured to the outer surface of the deformable band 402. In still another embodiment, the electronic system 404 is secured within the deformable band 402 with a portion of the system 404 exposed for user access, for example, to replace a battery or recharge the electronic system 404. In yet another embodiment, the electronic system 404 is attached to opposite ends of the deformable band 402 to complete a 360 degree enclosure. That is, opposite ends of the flexible, non-resilient deformable band 402 are attached to opposite ends of a housing including the electronic system 404 to complete the enclosure. In some embodiments, the deformable band 402 includes a textured outer surface, for example, ridges, bumps or other surface variations, to assist in securing hair within the device 400. The device 400 includes a central opening 419. According to some embodiments, the diameter of the central opening 419 is sized such that the device 400 can also be worn interchangeably as hair tie and as a wrist band while operating as an activity tracker.

Referring now to FIGS. 5A and 5B, a device 500 for activity tracking is illustrated in accordance with a fifth embodiment. The device 500 includes a hair band 502 and an electronic system 504. According to this embodiment, the device 500 includes a shell 510 including a first section 512 and a second section 514. According to one embodiment, the shell 510 is rigid, for example, manufactured from acrylic or another plastic. According to other embodiments, the shell 510 is a semi-rigid or flexible. According to the illustrated embodiment, the electronic system 504 is included in the first section 512. The respective ends of the two sections 512, 514 are attached to one another. A first attachment includes a hinge 516 and a second attachment includes a clasp 518 or other fastening element depending on the embodiment. According to other embodiments, a hinge may not be included if the material is sufficiently flexible such that the device 500 can be opened without the need for a separate hinge. The first attachment is located at the respective proximate ends of the two sections 512, 514. The second attachment is located at the respective distal ends of the two sections 512, 514. When secured to one another in a closed position with the clasp 518, the first section 512 and the second section 514 define a central opening 519. An inner surface 520 is located around the circumference of the central opening 519. According to the illustrated embodiment, a set of projections 522 extends radially inward from the inner surface 520.

In operation, the user releases the clasp 518 and opens the device 500 pivoting the first section 512 and the second section 514 apart using the hinge 516. The resulting operation is generally similar to the opening of a clam shell or a set of jaws. The user locates their hair within the central opening 519 and then closes the device securing the two sections 512, 514 to one another using the clasp 518. The set of projections 522 engages the strands of hair to secure the device 500 in place.

According to one embodiment, the set of projections 522 are manufactured from silicon. The type of projections employed can vary depending on the embodiment provided that the structure can engage the hair to securely attach the device 500 to the user's hair. According to one embodiment, the set of projections 522 includes a set of bristles. In other embodiments, the inner surface 520 includes the set of projections 522 that may be any of ridges, bumps or other texture either alone or in combination with a high friction material such as rubber. According to an alternate embodiment, the inner surface 520 is a smooth surface including a high friction material sufficient to grip the user's hair without any added projections.

In various embodiments, the electronic system 504 is located in either one of the first section 512 and the second section 514. According to the illustrated embodiment, the electronic system 504 is located within the first section 512 of the hair band 502. The rigid structure provided by the device 500 can allow the electronic system 504 to be located in a fixed location within the shell 510. Further, depending on the embodiment, the electronic system 504 can be housed in a manner that hides the system 504 from observers but allows the user to access the system 504 as necessary. According to one embodiment, the electronic system 504 is accessed via an exterior surface of the shell 510. According to another embodiment, the electronic system 504 is accessed via the surface 520 with the first section 512 and the second section 514 pivoted apart to place the device in an open position.

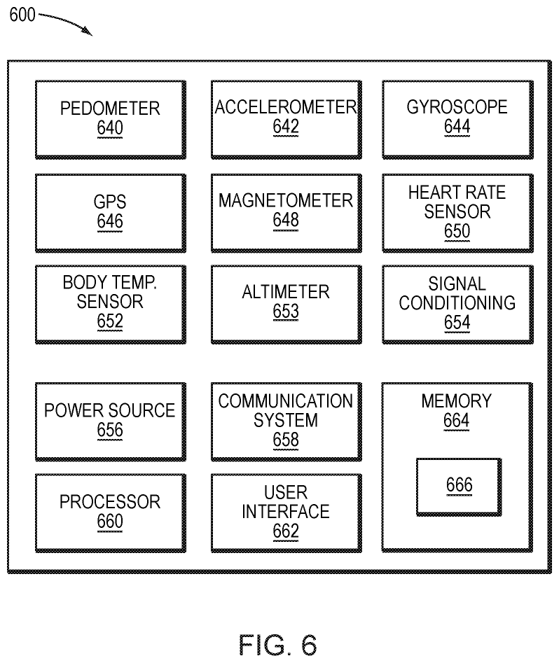

As described in greater detail below with reference to FIG. 6, the respective electronic systems 104, 204, 304, 404 each operate to record data concerning the activity of a user wearing the device 100, 200, 300, 400, respectively, as either a hair tie or a wrist band. The electronic system 504 operates to record data concerning the activity of a user wearing the device 500 as a hair tie. In various embodiments, the electronic system can include a processor, memory and sensors, for example, inertial sensors, biometric sensors or other sensors. In general, the respective electronic systems 104, 204, 304, 404, 504 measures and stores data from these sensors concerning the user's physical training, general level of activity and physiological condition. In various embodiments, the respective electronic systems 104, 204, 304, 404, 504 wireles sly communicate the data to another electronic device, for example, a smart phone or tablet computer for further processing and display of various metrics concerning the activity, health and well-being of the user. According to various embodiments, remote resources located over the “cloud” can receive the data for processing and/or storage. The preceding characteristics, including use in multiple configurations, also apply to the device 800 described below.

In addition, when used in a hair-tie configuration both the location and the central alignment of a hair tie relative to the user's head and torso when being worn provides particular advantages for activity tracking and health monitoring. For example, the electronic system can include contactless biometric sensors due to the close proximity to the users head and neck. The alignment above the spine can improve the accuracy and reliability of inertial measurements employed to measure a user's activity. In addition, the direct connection to the user's head provides the system with a unique ability to measure a user's head position, head tilt and overall posture.

In addition, the activity tracking devices illustrated and described herein are provided as a fashion accessory such as a hair tie or a wrist band. This allows the activity tracking device to be worn discretely because the electronic system can be hidden from view.

Referring now to FIG. 6, an electronic system 600 for inclusion in a fashion accessory is illustrated in accordance with some embodiments. According to the illustrated embodiment, the electronic system 600 includes a pedometer 640, an accelerometer 642, a gyroscope 644, a GPS 646, a magnetometer 648, a hear rate sensor 650, a body temperature sensor 652, an altimeter 653, signal processing circuitry 654, a power source 656, a communication system 658, a processor 660 (for example, a microcontroller), a user interface 662 and a memory 664.

The electronic system 600 includes one or more communication buses to connect the various components included in the system 600 to the processor 660, the memory 664 and/or one another as required by the application. The communication buses can be used for the communication of instructions/commands and data between the illustrated components and between the illustrated components and other components included in the device depending on the embodiment.

According to further embodiments, one or more elements of an overall system that includes the electronic system 600 can be provided in hardware or software included in a mobile device, for example, a tablet or smartphone. According to one version, at least a portion of the overall system is provided in a mobile app, for example, an app that receives information from the electronic system 600.

The memory 664 is configured to store software instructions 666 in accordance with various embodiments. In one embodiment, the memory 664 is included in the processor 660. In another embodiment, the memory 664 includes both memory internal to the processor 660 and memory external to the processor 660. In various embodiments, the memory 664 can include non-volatile memory, for example, EEPROM.

Depending on the embodiment, the power source 656 can include one or more batteries, for example, lithium or alkaline batteries. Further, the power source 656 can include a replaceable power source or a rechargeable power source depending on the embodiment. According to some embodiments, the electronic system 600 includes wireless charging circuitry, for example, inductive charging employed to recharge a rechargeable power source.

The user interface 662 can vary depending on the embodiment. According to one embodiment, the user interface 662 includes any one or any combination of indicating lights (for example, LEDs) to provide information concerning the operational status of the electronic system 600, for example, on/off, battery status and the status of wireless communication (i.e., whether the electronic system 600 successfully paired with a corresponding portable electronic device). According to a further embodiment, the user interface 662 can include one or more sensors to detect actions provided by the user, for example, the receipt of a tap “(or sequence of taps) on the electronic system 600 or housing in which the system 600 is provided. According to still further embodiments, the user interface 662 can include one or more feedback device. In one embodiment, the feedback device includes speakers to provide audio notifications or alerts. In another embodiment, the feedback device includes a tactile feedback device. For example, the feedback device can vibrate to provide notifications or alerts. According to one embodiment, the communication system 658 provides for local wireless communication via BLUETOOTH communication and/or long-distance communication, for example, via cellular and/or satellite communication networks. According to one embodiment, the communication system 658 provides BLUETOOTH communication suitable for transmission of information between the device 100, 200, 300, 400, 500 and a portable electronic device (for example, a mobile phone, tablet, etc.) located proximate to the device. According to this embodiment, a user can employ the device 100, 200, 300, 400, 500 in combination with a mobile phone or tablet to store user settings and data including data collected during one or more prior sessions in which the electronic system 600 is employed. In some further embodiments, the system 600 can also support hardwired communication systems in some embodiments, for example, communication via USB or other hardwired communication port included in the device 600.

The functionality of the electronic system 600 can vary depending on the embodiment. Accordingly, any of the pedometer 640, accelerometer 642, gyroscope 644, GPS 646, magnetometer 648, heart rate sensor 650, body temperature sensor 652 and altimeter 653 may be included on an optional basis depending on the functionality desired of a particular embodiment. Generally, however, the electronic system 600 will include at least one inertial measuring system, for example, at least one of the accelerometer 642 and the gyroscope 644. The accelerometer 642 can include a single axis accelerometer or a multi-axis accelerometer depending on the embodiment. In various embodiments, data from the accelerometer either alone or in combination with data from the gyroscope can be employed to determine step count, head position and posture of the user wearing the device 100, 200, 300, 400, 500. For example, the gyroscope 644 can be employed to record rotation of the head. According to a further embodiment, an inertial measurement unit including a multi-axis accelerometer and a gyroscope packaged in a single unit are provided in the electronic system 600.

According to another embodiment, the electronic system 600 includes a pedometer 640, for example, a pedometer chip. According to this embodiment, sensors integral to the pedometer 640 are employed to determine step count. In a further embodiment, the accelerometer 642 is employed in combination with the pedometer 640 to improve the accuracy of the step count.

In one embodiment, the GPS 646 included in the electronic system 600 is employed to determine both a current location and multiple locations between which the user has traveled. This data can be employed to determine a distance traveled by the user. The distance in combination with the elapsed time to travel the distance is used to determine the user's rate of speed, for example, a running or walking speed. According to a further embodiment, the GPS 646 is employed for locating the activity tracking system (and associated fashion accessory) if it is misplaced by the user. In some embodiments, an application operating on the user's portable electronic device allows the user to identify the location of the system.

In one embodiment, the magnetometer 648 included in the electronic system 600 is employed to provide directional information. For example, the magnetometer can be used to alert the user regarding a direction of travel or a deviation from a known and desired direction of travel.

In various embodiments, the heart rate sensor 650 samples the user's heart rate for use in computing the beats per minute. The heart rate sensor 650 includes a contactless system to accurately detect the user's heart rate without the need for the sensor 650 to contact the user's skin. According to one embodiment, the heart rate sensor 650 includes an optical system that senses the user's heart rate. According to an alternate embodiment, the heart rate sensor 650 includes an infrared hear rate sensor.

In various embodiments, the body temp sensor 652 includes a contactless system to accurately detect the user's body temperature with the need for the sensor 652 to contact the user's skin. According to one embodiment, the heart rate sensor 652 includes an infrared body temperature sensor.

In one embodiment, the altimeter 653 included in the electronic system 600 is employed to provide information concerning the altitude at which a user's activity is taking place. For example, the altimeter 653 can be used to evaluate the physical exertion and/or health benefits for training or other activities that occur at elevation where lower levels of oxygen are found.

Depending on the embodiment, the signal conditioning circuitry 654 can include one or more of analog-to-digital (A/D) converter, filters, amplifiers and other circuitry either alone or in combination with one another. According to one embodiment, the signal conditioning circuitry 654 operates to convert analog sensor output data to a digital format for further processing. According to another embodiment, at least some of the signal conditioning circuitry is included in the sensors themselves. According to one embodiment, each sensor includes integral signal processing where necessary and a discrete signal conditioning circuit is not employed.

In various embodiments, the device 100, 200, 300, 400, 500 including the electronic system 600 is employed in combination with a web or mobile application. Referring to FIG. 7, an activity tracking system 780 is employed by a user 781. According to the illustrated embodiment, the activity tracking system 780 includes an activity tracking device 782, for example, any of the activity tracking devices 100, 200, 300, 400, 500 illustrated and described herein, and a user device 784. According to the illustrated embodiment, the user device 784 includes a tablet computer. In general, the user device 784 can be any processing device whether mobile, laptop or stationary provided that the device is configured to access and operate the software application. According to further embodiments, the user device is a portable electronic device such as a smart phone or tablet.

In use, the sensor data is communicated from the activity tracking device 782 to the user device 784 for processing and display of activity and health related metrics. According to some embodiments, the sensor data itself is not displayed. Instead, the metrics are calculated using one or more algorithms that derive values from the sensor data. The metrics can include, step counts, heart rate data, body temp data, sleep-quality data, information on posture, distance traveled, calories burned and altitude as some examples. These metrics can be provided for a wide range of activity including pushups, crunches, squats, inversion, and other movements common in yoga, bane, Pilates, and dance. Metrics included on the app can also include user-provided information such as menstrual cycle or other personal health information. The software application that operates to process the data can provide the metrics in various formats, for example, as determined by user preferences. The format can included discrete values, average values, instantaneous values and may also include various forms of graphics. The graphics can include bar charts, pie charts, data plots over time and other approaches to clearly convey information to the user. The application can also provide a social aspect by allowing users to connect and share results with friends online.

According to a further embodiment, resources hosted in the cloud are employed. For example, in some embodiments, either or both of the activity tracking device 782 and the user device 784 communicate with cloud resources, not illustrated. For example, the sensor data can be communicated via a cellular data network to cloud resources. The data stored on the cloud can later be accessed by the user 701 with the user's device 784.

An axis A corresponding to an axis that aligns with a location of the user's spine is also illustrated in FIG. 7. The location of the activity tracking device 782 at the rear of the user's head can allow the electronic system 600 to be placed directly over the user's spine along the axis A. As described above, this “central” location can provide improved relevance and/or accuracy of sensor data measured by the electronic system 600. For example, the location of the activity tracking device 782 can be used to eliminate sensor data that reflects extraneous motion of the arms or other parts of the user's anatomy that might other be recorded using activity trackers worn on the wrist or other parts of the body.

In addition, the location can also allow the electronic system 600 to directly measure head position, head tilt, head motion and posture. The preceding cannot be accurately provided using prior approaches. For example, sensor data received from inertial sensors included in the activity tracking device 782 can be employed to record movement of the user's head when participating in athletics or training. This feedback can be evaluated to provide the user with information used to improve their performance. This feedback may also be evaluated to provide feedback on motions used in yoga, barre, dance, and other exercises with a significant floor component. Acceleration and direction can indicate when crunches, pushes, and inversions occur.

In various embodiments, the activity tracking device 782 can be provided in a form of any one of the devices 100, 200, 300, 400 and 800 described herein configurable for operation as an activity tracking fashion accessory that is configurable for use in multiple different configurations. In some of these embodiments, the activity tracking device 782 includes a resilient member sized to fit on a user's wrist in a first operating mode and for use as a hair tie in a second operating mode. These embodiments include an electronic system that is also configurable for activity tracking in each of the first operating mode and the second operating mode. With the activity tracking device 782 worn on a wrist of the user, the device 782 operates to measure both physiological parameters such as heart rate, pulse, body temperature and parameters concerning a movement of the user such as step-counts or other dynamic physical movement using, for example, one or more inertial sensors. With the activity tracking device 782 worn as a hair tie by the user, the device 782 can operate to measure parameters concerning a posture of the user in addition to either or both of the physiological parameters and the movement-parameters. The measurement of parameters can include a measurement of a single parameter or multiple parameters in any given category (i.e., posture, physiological, movement).

In some embodiments, the configurability of the electronic system occurs automatically, for example, where the electronic system detects a pattern of movement indicative of the activity tracking device being worn in a particular manner (i.e., on the wrist rather than as a hair tie). In other embodiments, the user employs their user device 784 to select one of the first operating mode or the second operating mode for the activity tracking device 782. This information is wirelessly communicated from the user device 784 to the activity tracking device 782. The electronic system included in the activity tracking device 782 operates by selectively adjusting one or more parameters that are measured based on the input received from the user and/or an automatic detection of the current manner in which the device 782 is being worn. In one approach, a measurement of one or more parameters can be selectively activated or deactivated based on the operating mode, for example, the posture parameter(s) may only be measured with the activity tracking device 782 worn as a hair tie. The manner in which a selected parameter is quantified based on measured data can also be modified depending on the operating mode. According to some embodiments, the signal processing of one or more sensor outputs is modified between the signaling processing performed when the activity tracking device 782 is worn as a wrist band and the signal processing performed when the device 782 is worn as a hair tie. This approach can be employed to maintain the required level of accuracy in any output values for a selected parameter despite the fact that the data from one or more selected sensors used to derive the output is differs significantly when the device 782 is worn in the different configurations.

According to some embodiments, the activity tracking device 782 can include visual indicia or a tactile indicators that assist the user in positioning the activity tracking device 782 such that the electronic system included therein is located on the underside of the bound-hair directly above the spine/neck. That is the indicia and/or indicators allow the user to rotate the hair tie to a desired rotational position about their bound-hair. This approach can assist the user by placing the contactless biometric sensors in close proximity to the user's exposed skin. For example, the activity tracking device 782 can include the electronic system in a manner that places the housing in which the electronic system is housed (for example, a metal or plastic enclosure that provides an electronic fob) proximate a skin surface of the user in each of a first operating mode (worn as a wrist band) and a second operating mode (worn as a hair tie). The preceding can allow the electronic system to better provide parameter-measurements concerning heart rate, pulse and/or body temperature. According to one embodiment, a clear plastic housing is provided in which the electronic system is located.

While shown and described with reference to devices 100, 200, 300, 400, 500, 800 embodiments of the activity tracking device 782 can include other styles and types of devices that are used to secure hair behind a user's head while also being configurable for wear as a wrist band or other fashion accessory. For example, the electronic system 600 can be included in a barrette in accordance with one embodiment.

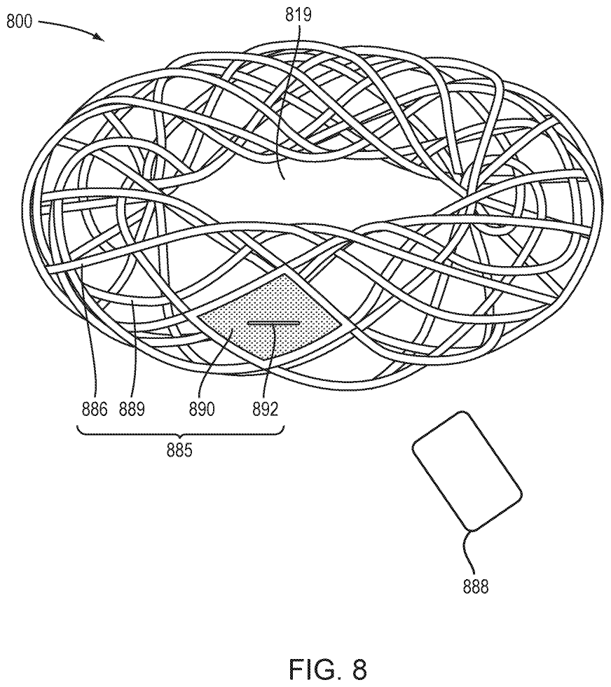

Referring now to FIG. 8, a device 800 for activity tracking is illustrated in accordance with another embodiment. In various embodiments, the device 800 is provided as a fashion accessory configurable for activity tracking when worn in any of a plurality of configurations, for example, as a hair tie and as a wrist band. According to the illustrated embodiment, the device 800 includes a resilient body 885 that includes bands 886 connected in a lattice structure that defines openings 889 in an overall tubular shape. That is, the resilient body 885 defines a hollow interior region. The resilient body 885 forms an overall toroidal shaped device that defines a central opening 819. The resilient body includes an enclosure 890 having an opening 892 to allow access to an interior of the enclosure 890. The device 800 also includes an electronic system 888 sized and configured to insert within the enclosure 890 via the opening 892. According to the illustrated embodiment, the electronic system is configured in the form of a fob. In this embodiment, the electronic system 888 can be housed in a housing manufactured of metal or plastic. Depending on the embodiment, the electronic system 888 can include any combination of the elements, features and functionality described with reference to the electronic system 600 of FIG. 6.

According to some embodiments, the device 800 is manufactured using a 3D printing process. Depending on the embodiment, other additive manufacturing processes can be used to manufacture the device 800. In general, the material of manufacture provides a resilient form to the lattice structure illustrated in FIG. 8 to allow the device 800 to be stretched over the hand for wear as a wrist band while also allowing the device 800 to be worn as a hair to secure the user's hair in a bundle. In one embodiment, a FIBERFLEX material is employed to manufacture the device 800 in a 3D printing process.

The central opening 819 can be sized to allow a user to slide their hand through the opening 819 to place the device 800 around their wrist. The device can also be worn as a hair tie in another configuration. According to some embodiments, the location of the enclosure 890 is such that the housing including the electronic system 888 is placed proximate a skin surface of the user with device 800 worn as either in a wrist band configuration or a hair tie configuration. According to these embodiments, the electronic system 888 is configured to measure at least one sensor output to provide data employed to quantify at least one of a heart rate of the user, a pulse of the user, and a body temperature of the user with the device 800 worn by the user in either of the first configuration or the second configuration.

Embodiments of the above-described devices 100, 200, 300, 400, 782 and 800 provide fashion accessory items that can be employed in multiple configurations by the user. In further embodiments, a plurality of resilient bands (for example, any of 102, 202, 302 and 885) can be included in a activity tracking fashion accessory kit that provides the user with a variety of multi-configuration fashion accessories in which an activity tacking electronic system can be interchangeably deployed. For example, in one embodiment, a plurality of the same type of one selected design can be provided in multiple colors (any one of the resilient band 102, the resilient band 202, the resilient band 302 and the resilient body 885). Each of the selected bands or body includes an enclosure (for example, the enclosure 890) configured to receive an electronic activity tracking system (for example, the electronic system 888 configured as a fob). The kit includes a single electronic activity tracking fob that can be interchangeable received in the enclosure included in each of the resilient elements included in a kit. Thus, a kit can include a plurality of resilient bands 102 as illustrated in FIG. 1. These bands can be provided in different colors and or patterns to make a different fashion statement depending on the band that is selected for wear. A user can select one of the bands, insert the electronic activity tracking fob and wear the band including the fob in any of multiple configurations for activity tracking. By swapping the electronic activity tracking fob between bands, the user may select a first band when going to the gym but another band when wearing the activity tracking fashion accessory to a wedding or other more formal event. In each configuration, the activity tracking fashion tracking accessory is not discernible as an activity tracking device. Instead, the activity tracking system appears as a conventional and stylish fashion accessory. According to another embodiment, the preceding can be achieved with a kit that includes a plurality of the different types of resilient members such as one each of the resilient band 102, the resilient band 202, the resilient band 302 and the resilient body 885 that can each be worn to accurately track activity in multiple configurations as a wrist band and as a hair tie.

The embodiments illustrated and described herein provide a device that is durable, waterproof and stretchable to allow wear as a configurable fashion accessory including an electronic activity tracking device. Depending on the embodiment, the resilient member included in the above described devices can be manufactured from any of rubber, foam, silicone or thermoplastic polyurethane. Embodiments are also manufactured using an additive manufacturing process. In addition to the preceding characteristics, these embodiments provide a configurable fashion accessory including an electronic activity tracking device that has a hi-coefficient of friction and a wide range of operating temperature across which the resilient member remains elastic and/or flexible. These features allow the device to provide a highly effective and long lasting hair tie in addition to use as a wrist band.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A system configurable for wear by a user in a plurality of locations for activity-tracking, the system comprising:
a resilient member having an overall shape that is substantially ring-shaped when in a relaxed state, the resilient member configurable in a plurality of states including the relaxed state and a plurality of different stretched states , the resilient member sized with an inner diameter smaller than a width of user's hand when in the relaxed state; and
an electronic system housed for secure attachment to the resilient member with the resilient member in any of the plurality of states, the electronic system configurable in a first operating mode for measuring with the resilient member worn on a wrist of the user a physiological parameter and a parameter concerning a movement of the user, the electronic system configurable in a second operating mode for measuring with the resilient member worn as a hair tie by the user at least two of the following parameters: the physiological parameter, the parameter concerning the movement of the user and a parameter concerning a posture of the user,
wherein the resilient member is deformable to place the resilient member into each of the plurality of different stretched states such that the overall shape of the resilient member in each the plurality of different stretched states includes non-circular shapes having inner diameters with dimensions that differ from the inner diameter of the resilient member in the relaxed-state. respectively,
wherein the electronic system is configured to receive a user input concerning a location on the user where the resilient member is secured, and
wherein the electronic system is configured to automatically record data measured for the parameter concerning the posture of the user when the user input indicates that the resilient member is being worn as the hair tie and automatically suspend a recording of data measured for the parameter concerning the posture of the user when the user input indicates that the resilient member is being worn as a wrist band.

2. The system of claim 1, wherein the resilient member includes an enclosure in which the electronic system is received, and
wherein the electronic system is housed within a fob sized and configured for insertion within the enclosure.

3. The system of claim 2, wherein the resilient member is configured to securely receive the fob in the enclosure in a substantially water-resistant manner

4. The system of claim 1, wherein the electronic system includes a wireless communication system configured to receive the user input communicated via a portable electronic device possessed by the user.

5. The system of claim 1, wherein the parameter concerning the movement of the user includes a parameter employed to track at least one of a number of steps taken by the user, a distance traveled by the user and a number of calories burned by the user, and
wherein data measured for the parameter concerning the movement of the user is available both when the resilient member is being worn as the hair tie and when the resilient member is being worn as the wrist band.

6. The system of claim 2, wherein the electronic system includes a rechargeable power source.

7. The system of claim 6, wherein the electronic system includes an electrical contact coupled to the rechargeable power source and accessible from an exterior of the resilient member with the fob received within the enclosure.

8. The system of claim 6, wherein the electronic system includes wireless charging circuitry.

9. The system of claim 1, wherein the resilient member includes a lattice structure configured in an overall shape that includes at least one of an overall toroidal-shape and an overall cylindrical-shape.

10. The system of claim 9, wherein the resilient member includes a fiberflex material.

11. An activity tracking fashion-accessory kit comprising:
an electronic system configured to measure parameters concerning physical activity when worn by a user; and
a plurality of resilient bands each including an enclosure configured to temporarily receive the electronic system in a manner that secures the electronic system within the resilient band, respectively, each one of the respective plurality of resilient bands sized and configured for use in each of a first configuration as a wrist band and a second configuration as a hair tie,
wherein the electronic system is housed in a housing that interchangeably fits within each of the respective enclosures, and
wherein the electronic system is configured to track a step count of the user when enclosed in any of the respective enclosures with the respective resilient band worn by the user in each of the first configuration and the second configuration, respectively.

12. The activity tracking fashion-accessory kit of claim 11, wherein a location of the enclosure in each of the respective resilient bands is configured to place the housing proximate a skin surface of the user with the respective resilient band worn by the user in either of the first configuration or the second configuration.

13. The activity tracking fashion-accessory kit of claim 12, wherein the electronic system is configured to measure at least one sensor output to provide data employed to quantify at least one of a heart rate of the user, a pulse of the user, and a body temperature of the user with the respective resilient band worn by the user in either of the first configuration or the second configuration.

14. The activity tracking fashion-accessory kit of claim 11, wherein the electronic system is configured to measure at least one sensor output to provide data employed to quantify steps taken by the user with the respective resilient band worn by the user in either of the first configuration or the second configuration.

15. The activity tracking fashion-accessory kit of claim 14, wherein the electronic system includes a wireless communication system configured to communicate the data to a personal electronic device in possession of the user.

16. A method of tracking a user's activity with a fashion accessory, the method comprising:
locating an activity tracking electronic system within a housing, the electronic system configured to wirelessly transmit sensor data concerning a movement of a user wearing the activity tracking electronic system to a portable electronic device in the user's possession;
providing a fashion accessory for use in each of a first configuration as a wrist band and a second configuration as a hair tie, the fashion accessory including an enclosure having an opening sized and configured to receive the housing;
inserting the housing within the enclosure via the opening;
in the first configuration, securing the fashion accessory around a wrist of the user in a manner that blocks both the opening and the housing from view; and
in the second configuration, securing the fashion accessory around a bundle of hair in a manner that blocks both the opening and the housing from view.

17. The method of tracking a user's activity with the fashion accessory of claim 16, further comprising configuring the fashion accessory to place the housing proximate a skin surface of the user with the respective resilient band worn by the user in either of the first configuration or the second configuration.

18. The method of tracking a user's activity with the fashion accessory of claim 17, further comprising measuring at least one sensor output to provide data employed to quantify at least one of a heart rate of the user, a pulse of the user, and a body temperature of the user with the fashion accessory worn by the user in either of the first configuration or the second configuration.

19. The method of tracking a user's activity with the fashion accessory of claim 16, further comprising quantifying steps taken by the user with the fashion accessory worn by the user in either of the first configuration or the second configuration.

20. The method of tracking a user's activity with the fashion accessory of claim 19, further comprising wirelessly communicating the data to a personal electronic device in possession of the user.
